(19) United States
(12) Patent Application Publication
(10) Pub. No.: US 2026/0290602 A1
(43) Pub. Date: Sep. 24, 2026

(54) METHOD AND COMPUTING DEVICE FOR QUANTIZING LEVEL OF CHRONIC INFLAMMATION

(71) Applicant: Kun Huei LAI, Taipei City (TW)

(72) Inventor: Kun Huei LAI, Taipei City (TW)

(21) Appl. No.: 19/436,579

(22) Filed: Dec. 30, 2025

(30) Foreign Application Priority Data
Mar. 18, 2025 (TW) 114110036

(51) Int. Cl. G16H 50/30; G16H 10/60

(52) U.S. Cl. CPC G16H 50/30; G16H 10/60

(57) ABSTRACT

A method for quantizing a level of chronic inflammation is adapted to be implemented by a computing device, and includes steps of, for one body part of a subject: a) obtaining multiple indicators; b) obtaining, for each of the indicators, a corresponding score that corresponds to the indicator, and determining, for each of multiple predefined adjustment conditions for the body part, whether the predefined adjustment condition for the body part is satisfied by at least one of the indicators; and c) performing, in a case where determination of step b) is affirmative, an adjustment process on the corresponding scores corresponding respectively to the indicators to obtain a total score that reflects a level of chronic inflammation related to the body part of the subject.

### CROSS-REFERENCE TO RELATED APPLICATION

This application claims priority to Taiwanese Invention Patent Application No. 114110036, filed on Mar. 18, 2025, the entire disclosure of which is incorporated by reference herein.

### FIELD

The disclosure relates to a method for quantizing a level of chronic inflammation occurring in a subject, and a computing device for quantizing a level of chronic inflammation occurring in a subject.

### BACKGROUND

Inflammation is a biological response of the body's immune system to defend against stimuli such as external stress or pathogens. If inflammation is prolonged, it will lead to excessive accumulations of reactive oxygen species (ROS) and free radicals in tissues, causing oxidative stress and damage, and eventually resulting in chronic inflammation. Chronic inflammation can occur in various organs and tissues of the body, and may cause eczema, urticaria, allergic rhinitis, allergic conjunctivitis, asthma, post-COVID-19 sequelae, autoimmune diseases, tumors, and so on.

Conventionally, diagnosis of chronic inflammation in clinical practice is carried out separately by doctors from different specialties, and different inflammatory symptoms presenting on different body parts are treated separately. However, such approach may ignore possible correlations among these different inflammatory symptoms, and may lead to uncomprehensive assessment and inappropriate treatment.

### SUMMARY

Therefore, an object of the disclosure is to provide a method for quantizing a level of chronic inflammation occurring in a subject, and a computing device for quantizing a level of chronic inflammation occurring in a subject.

According to one aspect of the disclosure, the method is adapted to be implemented by a computing device, and includes steps of, for one body part of the subject:
a) obtaining multiple indicators, each of the indicators being related to one of a symptom on the body part of the subject and a medication record of the subject;
b) obtaining, for each of the indicators, a corresponding score that corresponds to the indicator, and determining, for each of multiple predefined adjustment conditions for the body part, whether the predefined adjustment condition for the body part is satisfied by at least one of the indicators; and
c) performing, in a case where determination of step b) is affirmative, an adjustment process on the corresponding scores corresponding respectively to the indicators to obtain a total score that reflects a level of chronic inflammation related to the body part of the subject.

According to another aspect of the disclosure, the computing device includes a storage and a processor.

The storage stores multiple predefined adjustment conditions for one body part of the subject therein. Each of the predefined adjustment conditions is related to one of a specific symptom and a specific medication record.

The processor is electrically connected to the storage. The processor executes an operation procedure that includes steps of, for the body part of the subject,
a) obtaining multiple indicators, each of the indicators being related to one of a symptom on the body part of the subject and a medication record of the subject,
b) obtaining, for each of the indicators, a corresponding score that corresponds to the indicator, and determining, for each of the predefined adjustment conditions for the body part, whether the predefined adjustment condition for the body part is satisfied by at least one of the indicators, and
c) performing, in a case where determination of step b) is affirmative, an adjustment process on the corresponding scores corresponding respectively to the indicators to obtain a total score that reflects a level of chronic inflammation related to the body part of the subject.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating a computing device for quantizing a level of chronic inflammation occurring in a subject according to an embodiment of the disclosure.

FIGS. 2 and 3 are flow charts cooperatively illustrating a method for quantizing a level of chronic inflammation occurring in a subject according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

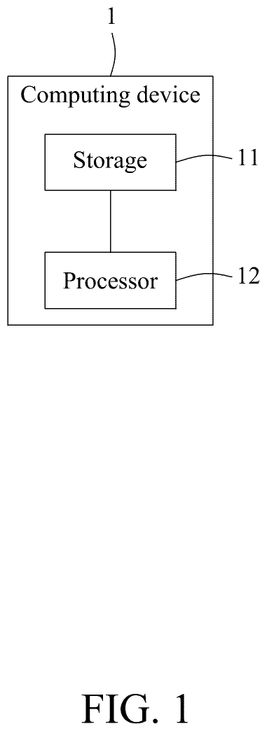

Referring to FIG. 1, an embodiment of a computing device 1 for quantizing a level of chronic inflammation occurring in a subject (e.g., a human) according to the disclosure is illustrated. The computing device 1 may be implemented to be a desktop computer, a laptop computer, a notebook computer or a tablet computer, but implementation thereof is not limited to what are disclosed herein and may vary in other embodiments. The computing device 1 includes a storage 11 and a processor 12 that are electrically connected to each other.

The storage 11 may be implemented by random access memory (RAM), double data rate synchronous dynamic random access memory (DDR SDRAM), read only memory (ROM), programmable ROM (PROM), flash memory, a hard disk drive (HDD), a solid state disk (SSD), electrically-erasable programmable read-only memory (EEPROM) or any other volatile/non-volatile memory devices, but is not limited thereto. The storage 11 stores multiple predefined adjustment conditions therein. Each of the predefined adjustment conditions is related to one of a specific symptom and a specific medication record. Each of the predefined adjustment conditions corresponds to a predefined weight. The predefined weights correspond respectively to the predefined adjustment conditions, and each of the predefined weights ranges from −1000 to +1000. In one embodiment, for each of the predefined adjustment conditions, the predefined weight is incorporated in the predefined adjustment condition, i.e., the predefined adjustment condition and the predefined weight are combined. In one embodiment, correspondence relationships between the predefined adjustment conditions and the predefined weights are recorded in a lookup table that is stored in the storage 11. It is worthy of note that the predefined adjustment conditions and the predefined weights corresponding thereto are crafted by medical professionals according to their experience and expertise.

The processor 12 may be implemented by a central processing unit (CPU), a microprocessor, a micro control unit (MCU), a system on a chip (SoC), or any circuit configurable/programmable in a software manner and/or hardware manner to implement functionalities discussed in this disclosure. The processor 12 is configured to obtain one of the predefined adjustment conditions stored in the storage 11, and to determine, for the one of the predefined adjustment conditions, the predefined weight corresponding to the one of the predefined adjustment conditions based on the one of the predefined adjustment conditions with which the predefined weight is combined or by looking up the lookup table stored in the storage 11.

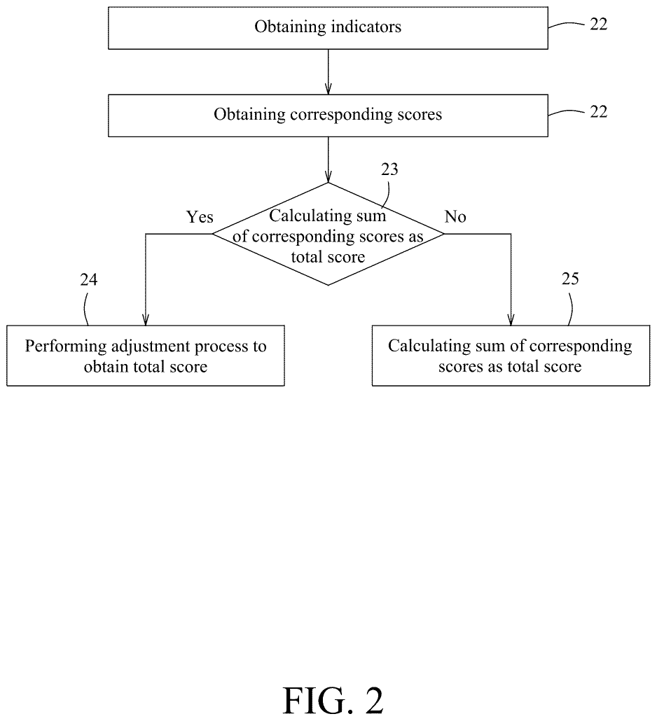

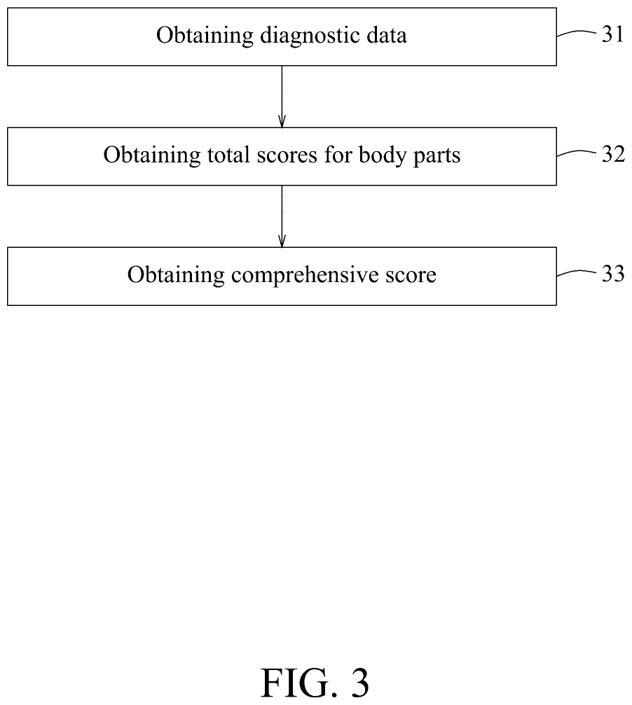

Referring to FIGS. 2 and 3, an embodiment of a method for quantizing a level of chronic inflammation occurring in a subject according to the disclosure is illustrated. The method is adapted to be implemented by the processor 12 of the computing device 1 that is previously described. The method includes steps 31 to 33 delineated below.

In step 31, the processor 12 obtains multiple entries of diagnostic data related to chronic illnesses of the subject. The entries of diagnostic data are exemplarily related to autoimmune diseases (e.g., “ANA,” “RF,” “Anti-ENA Ab,” “Anti-CCP,” “Anti-dsDNAAb,” “Anti-Cardiolipin IgG/IgM,” and so on), cancers (e.g., “CEA,” “AFP,” “CA 125,” “CA 19-9,” “CA 15-3,” “Cyfra 21-1,” “CA 72-4,” “SCC-Ag,” “EB-VCA IgA,” “PSA,” “Beta-HCG,” and so on), hyperplasia (e.g., gallbladder polyps, uterine fibroid, thyroid nodules, endometrioma, and so on), etc. Since the entries of diagnostic data have been well known to one skilled in the relevant art, detailed explanation of the same is omitted herein for the sake of brevity.

In step 32, for each of multiple body parts (e.g., skin, the nose, and so on) of the subject, the processor 12 executes an operation procedure to obtain multiple indicators for the body part, and to obtain a total score that reflects a level of chronic inflammation related to the body part of the subject. Specifically, for one body part of the subject, the operation procedure includes sub-steps 21 to 25 delineated below.

In sub-step 21, the processor 12 obtains multiple indicators. The indicators may be generated by a user (e.g., a medical professional) operating a keyboard (not shown) or a mouse (not shown) of the computing device 1 based on his/her observation of the subject, and may be implemented to contain text data, numerical data, date data, and so on. However, implementation of the indicators is not limited to the disclosure herein and may vary in other embodiments. Since implementation of obtaining the indicators has been well known to one skilled in the relevant art, detailed explanation of the same is omitted herein for the sake of brevity.

Each of the indicators is related to one of a symptom on the body part of the subject and a medication record of the subject. Specifically, the indicators include at least one of a first group of indicators that are related to visual features of a symptom experienced by the subject, a second group of indicators that are related to time features of a symptom experienced by the subject, or a third group of indicators that are related to a medicine taken by the subject.

More specifically, the first group of indicators include a primary-symptom indicator that indicates a primary symptom experienced by the subject, a scope indicator that indicates a scope of the primary symptom experienced by the subject, a complexity indicator that indicates a number of symptoms concurrently experienced by the subject, and a secondary-symptom indicator that indicates a secondary symptom experienced by the subject. The second group of indicators include a frequency indicator that indicates a frequency of onset of the primary symptom experienced by the subject, and a duration indicator that indicates a duration of onset of the primary symptom experienced by the subject. The third group of indicators include a drug-dependence indicator that indicates a degree of drug dependence of the subject, a multi-drug indicator that indicates a number of types of drugs used together by the subject, and an administration-route indicator that indicates administration route(s) of drug(s) used by the subject.

In sub-step 22, for each of the indicators, the processor 12 obtains a corresponding score that corresponds to the indicator. It should be noted that in this embodiment, correspondence relationships between the indicators and the corresponding scores are recorded in another lookup table that is stored in the storage 11, and the processor 12 is further configured to determine, for one of the indicators, the corresponding score that corresponds to the one of the indicators by looking up said another lookup table stored in the storage 11. The indicators and the corresponding scores are also crafted by medical professionals according to their experience and expertise.

For example, the processor 12 obtains a corresponding score of four when the primary-symptom indicator indicates the presence of hyperplasia, a corresponding score of two when the primary-symptom indicator indicates the presence of tissue swelling or rash, and a corresponding score of zero when the primary-symptom indicator indicates that the subject experiences no primary symptom. The processor 12 obtains a corresponding score of seven when the scope indicator indicates that the scope of the primary symptom experienced by the subject is about 70% of a body surface of the subject, a corresponding score of five when the scope indicator indicates that the scope of the primary symptom experienced by the subject is about 50% of the body surface of the subject, and a corresponding score of one when the scope indicator indicates that the scope of the primary symptom experienced by the subject is not greater than 10% of the body surface of the subject. The processor 12 obtains a corresponding score of two when the complexity indicator indicates that the subject experiences two symptoms concurrently, and obtains a corresponding score of one when the complexity indicator indicates that the subject experiences only one symptom. The processor 12 obtains a corresponding score of four when the secondary-symptom indicator indicates the presence of scleroderma in the skin of the subject, a corresponding score of one when the secondary-symptom indicator indicates the presence of tissue swelling or rash in the skin of the subject, a corresponding score of two when the secondary-symptom indicator indicates the presence of polyp in the nose of the subject, a corresponding score of one when the secondary-symptom indicator indicates the presence of tissue swelling or rash in the nose of the subject, and a corresponding score of zero when the secondary-symptom indicator indicates that the subject experiences no secondary symptom. The processor 12 obtains a corresponding score of seven when the frequency indicator indicates that the subject experiences the primary symptom seven days a week (i.e., every day), a corresponding score of two when the frequency indicator indicates that the subject experiences the primary symptom twice a week, and a corresponding score of one when the frequency indicator indicates that the subject experiences the primary symptom once a week. The processor 12 obtains a corresponding score of eight when the duration indicator indicates that a duration of onset of the primary symptom experienced by the subject is not less than twelve hours but less than twenty four hours, a corresponding score of four when the duration indicator indicates that a duration of onset of the primary symptom experienced by the subject is not less than four hours but less than twelve hours, and a corresponding score of two when the duration indicator indicates that a duration of onset of the primary symptom experienced by the subject is less than four hours. The processor 12 obtains a corresponding score of two when the drug-dependence indicator indicates that a degree of drug dependence of the subject is severe, and a corresponding score of one when the drug-dependence indicator indicates that the degree of drug dependence of the subject is mild. The processor 12 obtains a corresponding score of two when the multi-drug indicator indicates that the subject uses two types of drugs together, and a corresponding score of one when the multi-drug indicator indicates that the subject uses only one type of drug. The processor 12 obtains a corresponding score of two when the administration-route indicator indicates that an administration route of drug(s) used by the subject is an oral medication in a small amount, a corresponding score of four when the administration-route indicator indicates that an administration route of drug(s) used by the subject is an oral medication in a regular amount, a corresponding score of one when the administration-route indicator indicates that an administration route of drug(s) used by the subject is a topical medication, and a corresponding score of five (which could be interpreted as a sum of four and one) when the administration-route indicator indicates that an administration route of drug(s) used by the subject is a combination of the oral medication and the topical medication (no matter how many drugs are used).

In sub-step 23, for each of the predefined adjustment conditions for the body part, the processor 12 determines whether the predefined adjustment condition for the body part is satisfied by at least one of the indicators. In a case where the processor 12 determines that any one of the predefined adjustment conditions for the body part is satisfied by one of the indicators, a procedure flow of the method proceeds to sub-step 24. On the other hand, in a case where the processor 12 determines that none of the predefined adjustment conditions for the body part is satisfied by any one of the indicators, the procedure flow proceeds to sub-step 25.

In sub-step 24, the processor 12 performs an adjustment process on the corresponding scores corresponding respectively to the indicators to obtain the total score that reflects the level of chronic inflammation related to the body part of the subject.

In one embodiment of the adjustment process, the processor 12 calculates a summation of the corresponding scores corresponding respectively to the indicators as an initial score, and determines, for each of the indicators that satisfies one of the predefined adjustment conditions for the body part, whether the indicator is a to-be-subtracted indicator that satisfies one of multiple predefined subtraction conditions for the body part. In a case where it is determined that the indicator is the to-be-subtracted indicator, the processor 12 subtracts the corresponding score that corresponds to the to-be-subtracted indicator from the initial score, so as to obtain an adjusted initial score. Thereafter, for each of the predefined adjustment conditions for the body part that is satisfied by one of the indicators, the processor 12 multiplies the adjusted initial score (which is a result of subtracting all of the corresponding scores that correspond respectively to the to-be-subtracted indicators from the initial score) by the predefined weight corresponding to the predefined adjustment condition, so as to obtain the total score. That is to say, the total score is a product of the adjusted initial score and the predefined weights that correspond respectively to the predefined adjustment conditions which are satisfied.

In one embodiment of the adjustment process, the processor 12 calculates a product of the corresponding scores that correspond respectively to the indicators which satisfy the predefined adjustment conditions for the body part and the predefined weights that correspond respectively to the predefined adjustment conditions for the body part which are satisfied by the indicators so as to obtain a first sub-total score, and calculates a sum of the corresponding scores that correspond respectively to the indicators which do not satisfy any one of the predefined adjustment conditions for the body part to obtain a second sub-total score. Then, the processor 12 calculates a sum of the first sub-total score and the second sub-total score as the total score.

In sub-step 25, the processor 12 calculates a sum of the corresponding scores that correspond respectively to the indicators to obtain the total score that reflects the level of chronic inflammation related to the body part of the subject.

Subsequently, the processor 12 repeats execution of sub-steps 21 to 25 for another body part of the subject, so as to obtain multiple indicators for the another body part, and to obtain another total score that reflects a level of chronic inflammation related to the another body part of the subject.

In step 33, the processor 12 determines a comprehensive score that comprehensively reflects a level of chronic inflammation occurring in the subject based on the total scores obtained respectively for the multiple body parts of the subject. Then, the processor 12 adjusts the comprehensive score based on the entries of diagnostic data.

In a scenario where only one body part is considered, the processor 12 executes step 32 once, i.e., sub-steps 21 to 25 are executed only one time, and the processor 12 makes the total score obtained in step 32 serve as the comprehensive score in step 33.

In one embodiment where step 31 is omitted, the processor 12 does not adjust the comprehensive score based on the entries of diagnostic data in step 33.

It is worthy of note that an order of executing steps 31 and 32 is not limited to the disclosure herein and may vary in other embodiments. For example, in some embodiments, step 31 is executed in parallel with step 32 (i.e., steps 31 and 32 can be executed at the same time). In some embodiments, step 31 is executed after step 32.

It is worthy of note that in an allergen test report for a subject, an allergen-specific IgE level related to one kind of allergen shows a possibility of the subject being sensitive to that kind of allergen. An allergen-specific IgE level in grades 1 to 2 would contribute a reference score of one, an allergen-specific IgE level in grades 3 to 4 would contribute a reference score of ten, and an allergen-specific IgE level in grades 5 to 6 would contribute a reference score of twenty. A total reference score, which is independent of the comprehensive score, is determined based on grades of allergen-specific IgE levels in a comprehensive manner.

It should be noted that in practice, determination of the comprehensive score may take more conditions (e.g., interrelationships among indicators, special conditions caused by an illness, and so on) into account to improve accuracy of quantizing a level of chronic inflammation.

For explanation, several examples are provided in the following paragraphs.

A subject “A” is a severe allergy sufferer who suffers from eczema and allergic rhinitis according to indications from the indicators. Eczema flares up daily, and affects about 50% of a body surface area of the subject “A.” In addition, hardened and thickened skin (i.e., scleroderma) appears in the affected area of eczema. The subject “A” is on long-term medication (including oral and topical medications) to control eczema. Allergic rhinitis flares up about once a week. There are polyps in the nose of the subject “A.” The subject “A” takes a single kind of oral medication to control allergic rhinitis at the moment of being exposed to a specific allergen.

Corresponding scores and an initial score obtained by using the method according to the disclosure for each of two body parts (i.e., skin and the nose) of the subject “A” are summarized in Table 1 below.

TABLE 1

| | Corresponding score | |
|---|---|---|
| Indicator | Skin | Nose |
| Primary-symptom | 4 | 4 |
| indicator | (hyperplasia) | (hyperplasia) |
| Scope indicator | 5 | 1 |
| | (about 50% of body | (not greater than 10% |
| | surface) | of body surface) |
| Complexity indicator | 2 | 2 |
| | (two symptoms) | (two symptoms) |
| Secondary-symptom | 4 | 2 |
| indicator | (scleroderma) | (polyps) |
| Frequency indicator | 7 | 1 |
| | (daily) | (once a week) |
| Duration indicator | 8 | NULL |
| | (not less than 12 hours | |
| | but less than 24 hours) | |
| Drug-dependence | 2 | 1 |
| indicator | (severe) | (mild) |
| Multi-drug indicator | 2 | 1 |
| | (two types of drugs) | (one type of drug) |
| Administration-route | 5 | 4 |
| indicator | (oral and topical | (oral medication only) |
| | medications) | |
| Initial score | 39 | 16 |

Subsequently, the adjustment process is performed on the corresponding scores and the initial scores as shown in Table 1. Since the scope indicator for the skin satisfies a predefined adjustment condition for the skin (i.e., severity of the affected area reaches a predefined severity threshold) and the scope indicator for the skin is the to-be-subtracted indicator, the corresponding score of five is subtracted from the initial score for the skin, and the predefined weight of five will be used to obtain the total score for the skin later. Since the frequency indicator for the skin satisfies a predefined adjustment condition for the skin (i.e., severity of onset frequency reaches a predefined severity threshold) and the frequency indicator for the skin is the to-be-subtracted indicator, the corresponding score of seven is subtracted from the initial score for the skin, and the predefined weight of seven will be used to obtain the total score for the skin later. Since the duration indicator for the skin satisfies a predefined adjustment condition for the skin (i.e., severity of onset duration reaches a predefined severity threshold) and the duration indicator for the skin is the to-be-subtracted indicator, the corresponding score of eight is subtracted from the initial score for the skin, and the predefined weight of eight will be used to obtain the total score for the skin later. The drug-dependence indicator for the skin, the multi-drug indicator for the skin and the administration-route indicator cooperatively satisfy a predefined adjustment condition for the skin (which corresponds to the predefined weight of two) but none of them is the to-be-subtracted indicator, so only the predefined weight of two will be used to obtain the total score for the skin later. Consequently, the total score for the skin will be calculated as (39−5−7−8)×5×7×8×2=10640. The indicators for the nose do not satisfy any predefined adjustment condition, so the initial score (i.e., 16) for the nose would serve as the total score for the nose. Eventually, the comprehensive score for the subject “A” will be calculated as 10640+16=10656.

A subject “B” is also a severe allergy sufferer, and suffers from eczema and allergic rhinitis according to indications from the indicators. Eczema flares up daily, affecting approximately 70% of a body surface area of the subject “B.” Hardened and thickened skin appears in the affected area of eczema. The subject “B” is on long-term medication (including oral and topical medications) to control eczema. Allergic rhinitis flares up approximately once a week, and each time lasts for about four hours before spontaneously resolving. The subject “B” takes medication to control allergic rhinitis at the moment of being exposed to specific allergens. Furthermore, according to indication of diagnostic data, at a health check of the subject “B,” gallbladder polyps were found by using abdominal ultrasonography and uterine fibroids were found by using gynecologic ultrasonography. Allergen-specific IgE levels in an allergen test report show that the subject “B” is likely to be sensitive to three kinds of allergens in grades 5 to 6, and is likely to be sensitive to five kinds of allergens in grades 3 to 4.

Corresponding scores and an initial score obtained by using the method according to the disclosure for each of two body parts (i.e., skin and the nose) of the subject “B” are summarized in Table 2 below.

TABLE 2

| | Corresponding score | |
|---|---|---|
| Indicator | Skin | Nose |
| Primary-symptom | 4 | 2 |
| indicator | (hyperplasia) | (tissue swelling) |
| Scope indicator | 7 | 1 |
| | (about 70% of body | (not greater than 10% |
| | surface) | of body surface) |
| Complexity indicator | 2 | 1 |
| | (two symptoms) | (one symptom only) |
| Secondary-symptom | 4 | 1 |
| indicator | (scleroderma) | (tissue swelling) |
| Frequency indicator | 7 | 1 |
| | (daily) | (once a week) |
| Duration indicator | 8 | 2 |
| | (not less than 12 hours | (less than 4 hours) |
| | but less than 24 hours) | |
| Drug-dependence | 2 | 1 |
| indicator | (severe) | (mild) |
| Multi-drug indicator | 2 | 1 |
| | (two types of drugs) | (one type of drug) |
| Administration-route | 5 | 2 |
| indicator | (oral and topical | (oral medication in a |
| | medications) | small amount) |
| Initial score | 41 | 12 |

Subsequently, the adjustment process is performed on the corresponding scores and the initial scores as shown in Table 2. Since the scope indicator for the skin satisfies a predefined adjustment condition for the skin (i.e., severity of the affected area reaches a predefined severity threshold) and the scope indicator for the skin is the to-be-subtracted indicator, the corresponding score of seven is subtracted from the initial score for the skin, and the predefined weight of seven will be used to obtain the total score for the skin later. Since the frequency indicator for the skin satisfies a predefined adjustment condition for the skin (i.e., severity of onset frequency reaches a predefined severity threshold) and the frequency indicator for the skin is the to-be-subtracted indicator, the corresponding score of seven is subtracted from the initial score for the skin, and the predefined weight of seven will be used to obtain the total score for the skin later. Since the duration indicator for the skin satisfies a predefined adjustment condition for the skin (i.e., severity of onset duration reaches a predefined severity threshold) and the duration indicator for the skin is the to-be-subtracted indicator, the corresponding score of eight is subtracted from the initial score for the skin, and the predefined weight of eight will be used to obtain the total score for the skin later. Consequently, the total score for the skin will be calculated as (41−7−7−8)×7×7×8=7448. The indicators for the nose do not satisfy any predefined adjustment condition, so the initial score (i.e., 12) for the nose would serve as the total score for the nose. Then, the comprehensive score for the subject “B” will be temporarily calculated as 7448+12=7460.

Moreover, entries of diagnostic data related to hyperplasia show that gallbladder polyps contribute an additional score of nine (four scores of which are attributed to a primary symptom of gallbladder polyps, one score of which is attributed to a scope of gallbladder polyps, two scores of which are attributed to a number of concurrent symptoms, and two score of which is attributed to a secondary symptom of gallbladder polyps) on the comprehensive score, and uterine fibroids contribute an additional score of twelve (four scores of which are attributed to a primary symptom of gallbladder polyps, two scores of which are attributed to a scope of gallbladder polyps, two scores of which are attributed to a number of concurrent symptoms, and four scores of which are attributed to a secondary symptom of gallbladder polyps) on the comprehensive score. Irrelevant indicators are not taken into account. Therefore, the comprehensive score would be adjusted to be 7460+9+12=7481. The total reference score determined based on the allergen-specific IgE levels in the allergen test report ranges from ten to twenty.

A subject “C” is an allergy sufferer who only suffers from eczema according to indications from the indicators. Eczema flares up approximately twice a week. Each time when eczema flares up, eczema can be controlled simply by using a topical medication only. According to indication of diagnostic data, allergen-specific IgE levels in an allergen test report show that the subject “C” is likely to be sensitive to zero kinds of allergens in grades 5 to 6, and is likely to be sensitive to two kinds of allergens in grades 3 to 4.

Corresponding scores and an initial score obtained by using the method according to the disclosure for a body part (i.e., skin) of the subject “C” are summarized in Table 3 below.

TABLE 3

| | Corresponding score |
|---|---|
| Indicator | Skin |
| Primary-symptom | 2 |
| indicator | (tissue rash) |
| Scope indicator | NULL |
| Complexity indicator | 1 |
| | (one symptom only) |
| Secondary-symptom | 1 |
| indicator | (tissue rash) |
| Frequency indicator | 2 |
| | (twice a week) |
| Duration indicator | NULL |
| Drug-dependence | 1 |
| indicator | (mild) |
| Multi-drug indicator | 1 |
| | (one type of drug) |
| Administration-route | 1 |
| indicator | (topical medication only) |
| Initial score | 9 |

Since the indicators for the skin do not satisfy any predefined adjustment condition, the initial score (i.e., 9) for the skin would serve as the total score for the skin. Then, the comprehensive score for the subject “C” will be determined as nine.

Moreover, the total reference score is determined to be ten in a comprehensive manner based on the allergen-specific IgE levels in the allergen test report.

To sum up, for the method and the computing device for quantizing a level of chronic inflammation occurring in a subject according to the disclosure, in a scenario where chronic inflammation may affect only one body part of the subject, a corresponding score is obtained for each indicator that is related to a symptom on the body part or a medication record of the subject, and then a total score is calculated based on the corresponding score(s) obtained respectively for the indicator(s). The total score thus calculated reflects a level of chronic inflammation related to the subject. Moreover, in a scenario where chronic inflammation may affect multiple body parts of the subject, total scores are calculated in the aforementioned manner respectively for the body parts, and then a comprehensive score is determined based on the total scores thus calculated. Thereafter, the comprehensive score may be adjusted according to diagnostic data related to a chronic illness of the subject. The comprehensive score thus determined comprehensively reflects a level of chronic inflammation occurring in the subject. According to the comprehensive score, medical professionals may use appropriate treatments for chronic inflammation occurring in the subject.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for quantizing a level of chronic inflammation occurring in a subject, adapted to be implemented by a computing device, said method comprising steps of, for one body part of the subject:
a) obtaining multiple indicators, each of the indicators being related to one of a symptom on the body part of the subject and a medication record of the subject;
b) obtaining, for each of the indicators, a corresponding score that corresponds to the indicator, and determining, for each of multiple predefined adjustment conditions for the body part, whether the predefined adjustment condition for the body part is satisfied by at least one of the indicators; and
c) performing, in a case where determination of step b) is affirmative, an adjustment process on the corresponding scores corresponding respectively to the indicators to obtain a total score that reflects a level of chronic inflammation related to the body part of the subject.

2. The method as claimed in claim 1, each of the predefined adjustment conditions for the body part corresponding to a predefined weight, wherein step c) includes sub-steps of:
calculating a summation of the corresponding scores corresponding respectively to the indicators as an initial score;
determining, for each of the indicators that satisfies one of the predefined adjustment conditions for the body part, whether the indicator is a to-be-subtracted indicator that satisfies one of multiple predefined subtraction conditions for the body part;
subtracting, in a case where it is determined that the indicator is the to-be-subtracted indicator, the corresponding score that corresponds to the to-be-subtracted indicator from the initial score, so as to obtain an adjusted initial score; and
multiplying, for each of the predefined adjustment conditions for the body part that is satisfied by one of the indicators, the adjusted initial score by the predefined weight corresponding to the predefined adjustment condition, so as to obtain the total score.

3. The method as claimed in claim 1, each of the predefined adjustment conditions for the body part corresponding to a predefined weight, wherein step c) includes sub-steps of:
calculating a product of the corresponding scores that correspond respectively to the indicators which satisfy the predefined adjustment conditions for the body part and the predefined weights that correspond respectively to the predefined adjustment conditions for the body part which are satisfied by the indicators, so as to obtain a first sub-total score;
calculating a sum of the corresponding scores that correspond respectively to the indicators which do not satisfy any one of the predefined adjustment conditions for the body part, so as to obtain a second sub-total score; and
calculating a sum of the first sub-total score and the second sub-total score as the total score.

4. The method as claimed in claim 1, wherein the indicators include at least one of a first group of indicators that are related to visual features of a symptom experienced by the subject, a second group of indicators that are related to time features of a symptom experienced by the subject, or a third group of indicators that are related to a medicine taken by the subject.

5. The method as claimed in claim 4, wherein:
the first group of indicators include a primary-symptom indicator that indicates a primary symptom experienced by the subject, a scope indicator that indicates a scope of the primary symptom experienced by the subject, a complexity indicator that indicates a number of symptoms concurrently experienced by the subject, and a secondary-symptom indicator that indicates a secondary symptom experienced by the subject;
the second group of indicators include a frequency indicator that indicates a frequency of onset of the primary symptom experienced by the subject, and a duration indicator that indicates a duration of onset of the primary symptom experienced by the subject; and
the third group of indicators include a drug-dependence indicator that indicates a degree of drug dependence of the subject, a multi-drug indicator that indicates a number of types of drugs used together by the subject, and an administration-route indicator that indicates administration route(s) of drug(s) used by the subject.

6. The method as claimed in claim 1, further comprising, after step c) for the body part of the subject:
e) repeating execution of steps a) to c) for another body part of the subject, so as to obtain multiple indicators for the another body part, and to obtain another total score that reflects a level of chronic inflammation related to the another body part of the subject.

7. The method as claimed in claim 6, further comprising, after step e):
f) determining, based on the total score obtained for the body part and the another total score obtained for the another body part, a comprehensive score that comprehensively reflects a level of chronic inflammation occurring in the subject.

8. The method as claimed in claim 7, further comprising:
obtaining multiple entries of diagnostic data related to a chronic illness of the subject; and
after step f), adjusting the comprehensive score based on the entries of diagnostic data.

9. A computing device for quantizing a level of chronic inflammation occurring in a subject, said computing device comprising:
a storage that stores multiple predefined adjustment conditions for one body part of the subject therein, each of the predefined adjustment conditions being related to one of a specific symptom and a specific medication record; and
a processor that is electrically connected to said storage, and that executes an operation procedure, the operation procedure including steps of, for the body part of the subject,
a) obtaining multiple indicators, each of the indicators being related to one of a symptom on the body part of the subject and a medication record of the subject,
b) obtaining, for each of the indicators, a corresponding score that corresponds to the indicator, and determining, for each of the predefined adjustment conditions for the body part, whether the predefined adjustment condition for the body part is satisfied by at least one of the indicators, and
c) performing, in a case where determination of step b) is affirmative, an adjustment process on the corresponding scores corresponding respectively to the indicators to obtain a total score that reflects a level of chronic inflammation related to the body part of the subject.

10. The computing device as claimed in claim 9, each of the predefined adjustment conditions for the body part corresponding to a predefined weight, wherein step c) of the operation procedure includes sub-steps of:
calculating a summation of the corresponding scores corresponding respectively to the indicators as an initial score;
determining, for each of the indicators that satisfies one of the predefined adjustment conditions for the body part, whether the indicator is a to-be-subtracted indicator that satisfies one of multiple predefined subtraction conditions for the body part;
subtracting, in a case where it is determined that the indicator is the to-be-subtracted indicator, the corresponding score that corresponds to the to-be-subtracted indicator from the initial score, so as to obtain an adjusted initial score; and
multiplying, for each of the predefined adjustment conditions for the body part that is satisfied by one of the indicators, the adjusted initial score by the predefined weight corresponding to the predefined adjustment condition, so as to obtain the total score.

11. The computing device as claimed in claim 9, each of the predefined adjustment conditions for the body part corresponding to a predefined weight, wherein step c) of the operation procedure includes sub-steps of:
calculating a product of the corresponding scores that correspond respectively to the indicators which satisfy the predefined adjustment conditions for the body part and the predefined weights that correspond respectively to the predefined adjustment conditions for the body part which are satisfied by the indicators, so as to obtain a first sub-total score;
calculating a sum of the corresponding scores that correspond respectively to the indicators which do not satisfy any one of the predefined adjustment conditions for the body part, so as to obtain a second sub-total score; and
calculating a sum of the first sub-total score and the second sub-total score as the total score.

12. The computing device as claimed in claim 9, wherein the indicators include at least one of a first group of indicators that are related to visual features of a symptom experienced by the subject, a second group of indicators that are related to time features of a symptom experienced by the subject, or a third group of indicators that are related to a medicine taken by the subject.

13. The computing device as claimed in claim 12, wherein:
the first group of indicators include a primary-symptom indicator that indicates a primary symptom experienced by the subject, a scope indicator that indicates a scope of the primary symptom experienced by the subject, a complexity indicator that indicates a number of symptoms concurrently experienced by the subject, and a secondary-symptom indicator that indicates a secondary symptom experienced by the subject;
the second group of indicators include a frequency indicator that indicates a frequency of onset of the primary symptom experienced by the subject, and a duration indicator that indicates a duration of onset of the primary symptom experienced by the subject; and
the third group of indicators include a drug-dependence indicator that indicates a degree of drug dependence of the subject, a multi-drug indicator that indicates a number of types of drugs used together by the subject, and an administration-route indicator that indicates administration route(s) of drug(s) used by the subject.

14. The computing device as claimed in claim 9, wherein the operation procedure further includes, after step c) for the body part of the subject:
e) repeating execution of steps a) to c) for another body part of the subject, so as to obtain multiple indicators for the another body part, and to obtain another total score that reflects a level of chronic inflammation related to the another body part of the subject.

15. The computing device as claimed in claim 14, wherein the operation procedure further includes, after step e):
f) determining, based on the total score obtained for the body part and the another total score obtained for the another body part, a comprehensive score that comprehensively reflects a level of chronic inflammation occurring in the subject.

16. The computing device as claimed in claim 15, wherein the operation procedure further includes:
obtaining multiple entries of diagnostic data related to a chronic illness of the subject; and
after step f), adjusting the comprehensive score based on the entries of diagnostic data.
